(19) United States
(12) Patent Application Publication
(10) Pub. No.: US 2022/0096196 A1
(43) Pub. Date: Mar. 31, 2022

(54) THREE-DIMENSIONAL CRANIOCEREBRAL POSITIONING OPERATION TOOL AND METHOD OF USING THE SAME

(71) Applicants: Danian WEI, Guangzhou (CN); Haihua YANG, Guangzhou (CN)

(72) Inventors: Danian WEI, Guangzhou (CN); Haihua YANG, Guangzhou (CN)

(21) Appl. No.: 17/643,911

(22) Filed: Dec. 13, 2021

Related U.S. Application Data
(63) Continuation of application No. PCT/CN2020/102553, filed on Jul. 17, 2020.

(51) Int. Cl. A61B 90/14; A61B 90/00

(52) U.S. Cl. CPC A61B 90/14; A61B 2090/103; A61B 90/37; A61B 90/39

(57) ABSTRACT

A three-dimensional craniocerebral positioning operation tool and a use method thereof. The three-dimensional brain positioning operation tool and a use method thereof include a measurement method based on CT and MR images, a marking ruler and an bow-shaped guide device. The measurement method is used to measure the three-dimensional coordinate position of the focus, the marking ruler is used to draw the target point projection mark on the patient's body, and the bow-shaped guide device uses the projection mark to complete the lesion positioning. A spatial three-dimensional coordinate system can be established for CT or MR images of the patient's lesion through the corresponding software. The lesion positioning call be realized by using the the position of the lesion in the spatial three-dimensional coordinate system.

### CROSS REFERENCE TO RELATED APPLICATIONS

This application is a continuation of International Patent Application No. PCT/CN2020/102553 with a filing date of Jul. 17, 2020, designating the United States, now pending, and further claims priority to Chinese Patent Application No. 201910504055.9 with a filing date of Jun. 12, 2019. The content of the aforementioned applications, including any intervening amendments thereto, are incorporated herein by reference.

### TECHNICAL FIELD

The present disclosure relates to the field of medical marking positioning on surface of a human body, and more particularly relates to a three-dimensional craniocerebral positioning operation tool and a method of using the same.

### BACKGROUND

Existing positioning methods mainly include free-hand paracentesis, simple device-assisted guidance, traditional framed stereotaxic instrument, neuronavigation or surgical robot, etc. The free-hand paraoentesis method relies on simple tools to draw lines manually, and positioning bases on experience. It has an advantage of convenience and being fast. While, it has a poor accuracy, a high error mate, and it is greatly affected by human factors. The most important advantage of the traditional framed stereotaxic instrument and the neuronavigation system is the high positioning accuracy. However the operation process is very complicated, it is difficult to master, and the equipment is expensive. In addition to the above two, methods, there are special surgical robots for brain surgery, such, as the ROSA developed by the French company Medtech SA and the domestic Ruimi robot. The advantage of the surgical robot is precision and intelligence, but the price is too high and it is difficult to popularize.

Thea main defects of the above methods are as follows: 1. The measurement and marking of the target site are either too rough or the process is too complicated; 2. Positioning operations depend either on experience or complex equipment. Convenience, accuracy and cost performance cannot be balanced.

### SUMMARY

1. Technical Problem to be Solved

In order to solve the problem that the measurement and marking of the target site are either too rough or the process is too complicated, the present disclosure provides a new method. The anatomical coordinate system is corresponded to the image coordinate system by using the distinguishable anatomical landmarks of the human body surface. Two sets of unified measurement coordinate systems having Corresponding origin positions and coordinate axis directions (the horizontal direction is X, the vertical direction is Y, and the up or down direction is Z) are set up, which are respectively called the image measurement coordinate system on the image and the anatomical measurement coordinate system on the human body. In such a way, the imaging measurement and anthropometric measurement can be unified For a head, as plane passing through the orbitomeatal base line (a connecting line of an external auditory canal and a midpoint of an eyeball) is used as the horizontal plane of the measurement coordinate system, and the midsagittal plane is used as the sagittal plane of the measurement coordinate system. The coronal plane passing through the external auditory canal and perpendicular to the above two planes is used as the coronal plane of the measurement coordinate system. In this way, the imaging and anatomy coordinate systems are consistent, and each point in the cerebral space corresponds to the same and unique three-dimensional coordinate values in the two three-dimensional coordinate systems. A new three-dimensional coordinate system is adopted, the image coordinates and anatomical coordinates are unified. A new method that can realize software automatic measurement of the target point is designed by using the characteristics of CT and MR images, avoiding the installation of the base ring and the review of CT or MR, pasting Marker, copying DICOM image data and other tedious processes, making image measurement easier and faster.

In order to solve the problem of inconvenient head marking, the present disclosure provides a specially designed marking ruler to quickly mark the projection position of the target point on the body surface.

In view of the problem that the positioning operations depend either on experience or complex equipment, the present disclosure provides two three-dimensional guide devices which are simple in structures, convenient to install, multifunctional, and do not affect the operation area. The two three-dimensional guide devices can complete most of the brain positioning functions, greatly reduce the complexity of positioning operations without affecting the accuracy.

The disclosure reduces the pre-operation preparation time and the operation tune during the operation from multiple links, the operation is more scientific, the positioning is more accurate, the time is saved, the operation cost is reduced, and the operation effect is improved.

### 2. Technical Solutions

To solve the above-mentioned problems the present disclosure adopts the following technical solutions.

Technology solution of target measurement.

Steps:

(1) setting a two-dimensional coordinate system (reference coordinate) on a cross sectional image by using a proportional scale; wherein an origin of the two-dimensional coordinate system can be any point on the proportional scale (preferably a starting point or an end point), and a coordinate origin on each cross-section must be consistent. The coordinate scale is the same as the proportional which represents the actual length (usually 1 cm):

(2) selecting a piece of axial CT or MRI showing an external auditor; canals crystalline lenses on both sides, determining at midpoint of a connecting line (X axis) of the external auditory canals on both sides, and measuring a two-dimensional coordinate value of this point (the origin of the image measurement coordinates) in the above reference coordinate based on the proportional scale: establishing the reference coordinate by using this point as the origin: wherein a vertical axis (Y axis) is consistent with a midsagittal line of a head (normally, it is straight up and down, if there is a tilt, it needs to be, rotated accordingly, and the rotation angle ∠ω is recorded);

(3) selecting another cross-section plane where the target is located, establishing a reference coordinate by using the same method based on the position and scale of the proportional scale, and marking the origin coordinate value of the measurement coordinate obtained in the previous step in the reference coordinate of the another cross-section plane: and establishing another image measurement coordinate for the origin of the another cross-section plane by the origin coordinate value; wherein the scale of this two-dimensional coordinate is the same as the reference coordinate; the direction of the vertical axis is also consistent with the midsagittal line of the head, and the rotation angle is the same as ∠ω;

(4) At this time, any point on the image can be positioned within this image measurement coordinate, and the abscissa (X) and ordinate (Y) of this point can be measured, as well as the angle of the long axis of the focus relative to the measurement coordinate;

(5) calculating a distance (Z) between the target point and the baseline plane based on the layer thickness and the number of layers frons the target point plane to the baseline plane;

(6) finally determining a three-dimensional coordinate value (X/Y/Z) for any point in the head.

Software can be used for automatical measurement in practical applications. Steps are as follows:

Step 1. taking pictures of the CT or MR image data, selecting a scanning plane (baseline plane) that shows the external auditory canals and crystalline lenses, creating a virtual proportional scale on the software, and overlapping the length and position of the virtual proportional scale with that of the proportional scale on the image data by manual or automatic recognition methods to obtain a scaling of the pictures, and at the same time using the position of the proportional scale on the image data as the reference object; in this way, the extraction of image data is realized, and the next step of coordinate positioning and length measurement can be carried out;

Step 2. drawing the connecting line of the midpoints f the external auditory canals on both sides and the mid-sagittal line manually or through the automatic recognition function of the software to form a cross line after the position is determined; using the intersection of the cross line as the origin of the measurement coordinates: measuring the relative position coordinates of this point relative to the proportional scale (reference coordinates) used as the reference object in step 1; if the picture is rotated, the cross line can be rotated for correction, and the rotation angle will be recorded by the software;

Step 3. selecting the scanning plane were the target to be positioned is located (the same photo or re-photographed photos cast be used), and displaying the virtual proportional settle on the software again, overlapping the virtual proportional scale with the proportional scale on the current image data by manual or automatic recognition function; calculating by software to automatically generate a two-dimensional coordinate with the coordinate Value of the intersection point as a center by using the two-dimensional coordinates of the intersection of the cross line in the reference coordinates and the rotation angle of the cross line; at this time, the generated two-dimensional coordinate is the measurement coordinate, the position of the measurement coordinate on the target plane is consistent with the position of the above cross line on the baseline plane;

Step 4. measuring the position of all point in the target plane by using the two-dimensional measurement coordinate generated in step 3, and clicking on any position to display the two-dimensional coordinate values;

Step 5. drawing a straight line with the target point as the starting point, and measuring an angle between the straight line and the two coordinate lines through the software, if the direction of the path to reach this point needs to be measured.

Technology solution of marking ruler.

The external auditory canal is used for marking of the target point on human body surface. The marking ruler includes a fixed marking ruler and a foldable marking ruler, the fixed marking ruler comprises a fixed horizontal ruler; and a fixed Z-axis ruler is fixedly connected to one end of the fixed horizontal ruler, and the fixed Z-axis ruler and the fixed horizontal ruler are perpendicular to each other; the fixed Z-axis ruler and the fixed horizontal ruler are both engraved with scale marks; zero-scale ends of the fixed Z-axis ruler and the fixed horizontal ruler are located at an intersection of the fixed Z-axis ruler and the fixed horizontal ruler; a main slider is sleeved on the fixed horizontal ruler, and the fixed horizontal ruler is matched with the main slider; the main slider is capable of moving slowly on the fixed horizontal ruler under an action of external force.

Further, the marking ruler may be a foldable marking ruler, the foldable marking ruler comprises a foldable horizontal ruler; one end of the foldable horizontal ruler is connected to a foldable Z-axis ruler, and the foldable Z-axis ruler is perpendicular to the foldable horizontal ruler: the foldable Z-axis ruler and the foldable horizontal ruler are both engraved with scale marks: zero-scale ends of the foldable Z-axis ruler and the foldable horizontal ruler are located at an intersection of the foldable Z-axis ruler and the foldable horizontal ruler; an L-shaped groove is formed on an end of the foldable Z-axis ruler close to the foldable horizontal ruler; an end of the foldable horizontal ruler close to the foldable Z-axis ruler is fixedly provided with two triangular plates, and the two triangular plates are respectively located on two sides of the foldable Z-axis ruler; a connecting shaft is fixedly connected between the two triangular plates, and the connecting shaft penetrates the L-shaped groove; the foldable Z-axis ruler and the foldable horizontal ruler are movably connected through the L-shaped groove and the connecting shaft. It is convenient for the medical staff to carry it daily, so that the marking ruler is not easy to be damaged by accidental bumps due to its excessive size.

Technology solution of three-dimensional guide device.

The quarter-bow guide device comprises a first quarter-h guide device; and the first quarter-bow guide device comprises a first main fixing frame and a first accessory fixing frame; a main square slot that fits the first accessory fixing frame is formed in the first main fixing frame, and the first accessory fixing frame is inserted in the main square slot; a main through hole slot is formed in an upper end of the first main fixing frame, and a main knob is inserted in the main through hole slot; and upper end of the first accessory fixing frame is fixedly connected with a main serrated block that fits the main knob: and the first main fixing frame and the first accessory fixing frame are fixedly connected by the main serrated block and the main knob therebetween; lower ends of the first main fixing frame and the first accessory fixing frame are fixedly connected with main head nails; and a side wall of the first main fixing frame is fixedly connected with a main connecting block; and one end of the main connecting block far away from the first main fixing frame is fixedly connected with a main quarter-bow: and a main positioning device is slidably connected on the main quarter-bow; and a main zero-degree midpoint is engraved on the main connecting block.

Further, the quarter-bow guide device comprises a second quarter-bow guide dev ice, and the second quarter-bow guide device comprises a second main fixing frame and a second accessory fixing frame: an accessory square hole slot that fits the second accessory fixing frame is formed in the second main fixing fame, and the second accessory fixing frame is inserted into the accessory square hole slot: a accessory through hole slot is formed in an upper end of the second main fixing frame, and upper end of the second main fixing frame is connected with an accessory knob; and the accessory knob penetrates the accessory through hole slot, and an upper end of the second main fixing frame is fixedly connected with an accessory serrated block that matches with the accessory knob; lower ends of the second main fixing frame and the second accessory fixing frame are respective fixedly connected with a short head nail and a long head nail; a sliding connection block is slidably connected to the long head nail, and the sliding connection block is located at an end of the long head nail away from the short head nail; the sliding connection block, is fixedly connected with an accessory quarter-bow, the accessory quarter-bow is slidably connected with an accessory positioning device, an accessory zero-degree midpoint is engraved on the accessory quarter-bow. The second quarter-bow guide device can assist in target positioning.

Further, an inner diameter and an outer diameter of main quarter-bow and the accessory quarter-bow are 180 mm-300 mm and 220 mm-400 mm respectively; and a width of the main quarter-bow and the accessory quarter-bow is 20 mm-50 mm; such that they can be applied to patients of different body types.

Further, a material, of the main quarter-bow and the accessory quarter-bow is selected from metal, plastic and polymer material; and tips of the main quarter-bow and the accessory quarter-bow are polished smoothly, so that the main quarter-bow and the accessory quarter-bow are not easy to paracentesis the patient.

Further, the main positioning device and the accessory positioning device have same structure: each of the main positioning device and the positioning devices comprises a positioning block: one end of the positioning block is engraved with a rotational sliding groove: and the positioning block is connected to the main quarter-bow or the accessory quarter-bow through the rotational sliding groove; and a slot is formed at an end of the positioning block away from the rotational sliding groove. It is convenient for the medical staff to slide the positioning block on the main quarter-bow or the accessory quarter-bow.

Further, an extension rod is inserted into the slot: and the extension rod comprises an L-shaped rod body: the L-shaped rod body comprises a main body part and an extension part; the extension part of the L-shaped rod body is fixedly connected with a plug-in block: and a positioning hole is formed in the plug-in block; and an axis of the positioning, hole is parallel to the main body part of the L-shaped rod body. The main body part has a length of 10-15 cm and a trapezoidal cross-section, and it can be inserted into the slot on the positioning block. The main body part has a scale on it, which can correspond to the paracentesis depth; and the extension part has a length of 3-5 cm.

Further, a pair of guide channels matches with each other are inserted into the positioning hole, and one end of the guide channel is fixedly connected a limited block. The two guide channels form a complete guide channel for guiding. The main body part of the L-shaped rod body is inserted into the slot in the positioning block, the insertion depth is adjusted as required: a pair of guide channels are inserted into the positioning hole, and the channel centers of the guide channels are aligned with the center of the semicircular-bow, the extension rod can slide on the semicircular-bow with the positioning block, and always the center points to the center of the semicircular-bow.

Further, the guide channel is inserted into a half-column groove, and the guide channel has a variety of models, and different models of the half-column groove have different diameters, which can be matched with guide channels with diameters of different sizes.

Further, the using method of the three-dimensional brain positioning operation tool is as follows:

S1. filming CT or MR data images, and transmitting the data images obtained to a computer or other processing terminal, and transferring the data images to corresponding processing software;

The specific steps are as follows:

(1) selecting a reference coordinate selecting a scanning plane (baseline plane) that shows the external auditory canals and eve lenses, creating a virtual scale ran the software, and overlapping the length and position of the scale on the image data by manual or automatic recognition methods to obtain the scaling of the pictures, and at the same time using the position of the scale on the image data as the reference object:

(2) determining the relative coordinates; drawing the connecting line of the midpoints of the external auditory canals on both sides and the mid-sagittal line manually or through the automatic recognition function of the software to form a cross line after the position is determined; using the intersection of the cross line as the origin of the measurement coordinates; measuring, the relative position coordinates of this point relative to the proportional scale (reference coordinates) used as the reference object in step 1: specially, if the picture is rotated, the cross line can be rotated for correction, and the rotation angle will be recorded by the software;

(3) determining a two-dimensional coordinates of the focus; selecting the scanning plane where the target to be positioned is located (the same photo or re-photographed photos can be used), and displaying the Virtual proportional scale on the software again, overlapping the virtual proportional scale with the proportional scale on the current image data by manual or automatic recognition function: calculating by software to automatically generate a two-dimensional coordinate with the coordinate value of the intersection point as a center by using the two-dimensional coordinates of the intersection of the cross line in the reference coordinates and the rotation angle of the cross line; at this time, the generated two-dimensional coordinate is the measurement coordinate, the position of the measurement coordinate on the target plane is consistent with the position of the above cross line on the baseline plane;

(4) determining the two-dimensional coordinate value of the focus; measuring the position of any point in the target plane by using the two-dimensional measurement coordinate generated in step 3, and clicking on any position to display the two-dimensional coordinate values;

S2. firstly drawing a scanning baseline on a body surface; the scanning baseline being an orbitomeatal base line which is a connecting line of an external auditory canal and a midpoint of an eyeball; and then marking two epieranium projection points, a midline and parallel lines beside the midline of a head by using the marking ruler;

S3. determining a paracentesis point by using the quarter-bow guide device.

Beneficial Effect.

Compared with the prior art, the advantages of the present disclosure are as follows.

The present disclosure has the following beneficial effects:

1. A new three-dimensional coordinate system is adopted, the image coordinates and anatomical coordinates are unified.

2. A new method that can realize software automatic measurement of the target point is designed by using the characteristics of CT and MR images, avoiding the installation of the base ring and the review of CT or MR, pasting Marker, copying DICOM image data and other tedious processes, making image measurement easier and faster.

3. The present disclosure provides a specially designed marking ruler to quickly mark the projection position of the target point on the body surface.

4. The three-dimensional guide devices are simple in structures, convenient to install, multifunctional, and, do not affect the operation area. The two three-dimensional guide devices can complete most of the brain positioning functions.

5. The pre-operation preparation time and the operation time during the operation are reduced from multiple links, the operation is more scientific, the positioning is more accurate.

6. The complexity of positioning operations is greatly reduced without affecting the accuracy, and the time is saved, the operation cost is reduced, and the operation effect is improved.

### BRIEF DESCRIPTION OF THE DRAWINGS

FIG. 1 is a front view of the first quarter-bow guide device of the present disclosure;

FIG. 2 is a side cross-sectional view of the first quarter-bow guide device of the present disclosure;

FIG. 3 is a front view of the second quarter-bow guide device of the present disclosure;

FIG. 4 is a front cross-sectional view of a joint between the second main fixing frame and the second accessory fixing frame of the second quarter-bow of the present disclosure:

FIG. 5 is a structural schematic diagram of the positioning slider of the quarter-bow guide device oaf the present disclosure;

FIG. 6 is a structural schematic diagram of the guide channel of the quarter-bow guide device of the present disclosure;

FIG. 7 is a front view of the extension rod of the present disclosure;

FIG. 8 is a structural schematic diagram showing the extension red of the present disclosure inserted into the existing quarter-bow guide device;

FIG. 9 is a front view of the non-foldable marking ruler of the present disclosure;

FIG. 10 is a front view of the foldable marking ruler of the present disclosure;

FIG. 11 is a schematic diagram of the establishment of reference coordinates of the present disclosure;

FIG. 12 is a schematic diagram of the establishment of target coordinates of the present disclosure:

FIG. 13 is a front view of the first semicircular-bow guide device of the present disclosure;

FIG. 14 is a front view of the second semicircular-bow guide device of the present disclosure;

FIG. 15 is a sectional view of the positioning nail in FIG. 8 of the present disclosure;

FIG. 16 is a structural schematic diagram of another embodiment of the present disclosure showing the extension rod is inserted into the existing quarter-bow guide device;

FIG. 17 is a front view of a foldable marking ruler according to another embodiment of the present disclosure:

FIG. 18 is a top view of the foldable marking ruler according to another embodiment of the present disclosure;

FIG. 19 is a schematic diagram of an application scenario of the foldable marking ruler according to another embodiment of the present disclosure:

FIG. 20 is a schematic diagram showing a use state of the application scenario of FIG. 19 in another direction;

FIG. 21 is a schematic diagram showing the first semicircular-bow guide device of the present disclosure applied to cranial positioning;

FIG. 22 is a schematic diagram showing the second semicircular-bow guide device of the present disclosure applied to cranial positioning.

Explanation of reference signs in the figures:

101, first main fixing frame; 102, first accessory fixing frame; 103, main head nail; 104, main connecting block; 105, main quarter-bow; 106 main knob; 107, main zero-degree midpoint; 108, main positioning device; 201, second main fixing frame; 202, second accessory fixing frame; 203, short head nail; 204, long head nail; 205, accessory square hole slot; 206, accessory through hole slot; 207, accessory knob; 208, accessory serrated block; 209, sliding connection block; 210 accessory quarter-bow; 211, accessory positioning device: 212, accessory zero-degree midpoint; 301, fixed Z-axis ruler; 302, fixed horizontal ruler: 303, main slider: 401, foldable Z-axis ruler: 402, foldable horizontal ruler: 403. L-shaped groove; 404, triangular plate: 405, connecting shaft:, 5, positioning block; 6, rotational sliding groove: 7, slot; 8, guide channel: 9, limited blocks; 10, extension rod: 1001, L-shaped rod body: 1002, plug-in block; 1003, positioning hole.

### DETAILED DESCRIPTION OF PREFERRED EMBODIMENTS

The technical solutions in the embodiments of the present disclosure will be clearly and completely described below in conjunction with the accompanying drawings. Obviously, the described embodiments are only a part of the embodiments of the present disclosure, rather than all the embodiments. Based on the embodiments of the disclosure, all other embodiments obtained by those of ordinary skill in the art without creative work shall fall within the protection scope of the present disclosure.

In the description of the present disclosure, it should be noted that the directions or positional relationships indicated by the terms “upper”, “lower”, “inner”, “outer” and “top/bottom” are based on the directions shown in the drawings. The positional relationship is only for the convenience of describing the present disclosure and simplifying the description, rather than indicating or implying that the pointed device or element must have a specific: orientation, be constructed and operated in the specific orientation. Therefore it cannot be understood as a limitation of the present disclosure. In addition, the terms “first” and “second” are only used for descriptive purposes, and cannot be understood as indicating or implying relative importance.

In the description of the present disclosure, it should be noted that, unless otherwise clearly specified and limited, the terms “installed”, “provided with”, “sleeved/connected”, “connection”, etc., should be understood in a broad sense. For example, “connection” can be a fixed connection, a detachable connection, or an integral connection; it also can be a mechanical connection or an electrical connection; it also can be direct connection, indirect connection through an intermediate medium, gar connection within two components. For those of ordinary skill in the art, the specific meaning of the above-mentioned terms in the present disclosure can be understood in specific situations.

### Embodiment 1

The three-dimensional brain positioning operation tool, its use method is:

S1. filming CT or MR data images, and transmitting the data images obtained to a computer or other processing terminal, and transferring the data images to corresponding processing software;

The specific steps are as follows:

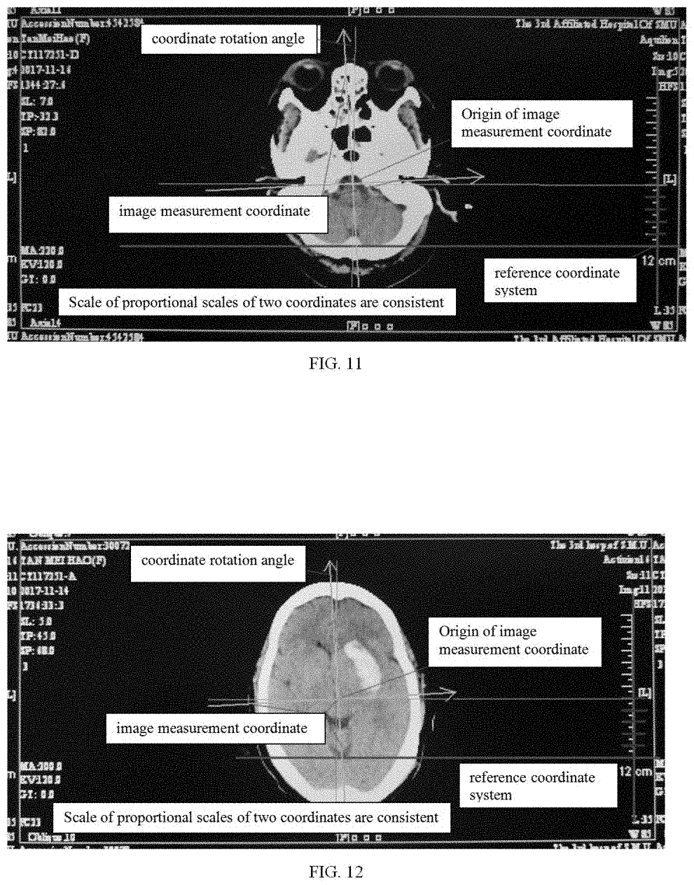

(1) selecting a reference coordinate; selecting a scanning plane (baseline plane) that shows the external auditory canals and crystalline lenses, creating a virtual proportional scale on the software, and overlapping the length and position of the virtual proportional scale with that of the proportional scale on the image data by manual or automatic recognition methods to obtain a scaling of the pictures, and at the same time using the position of the proportional scale on the image data as the reference object please refer to FIG. 11;

(2) determining the relative coordinates; drawing the connecting line of the midpoints of the external auditory canals on both sides and the mid-sagittal line manually or through the automatic recognition function of the software to Suing a cross line after the position is determined; using the intersection of the cross line as the origin of the measurement coordinates-, measuring the relative position coordinates of this point relative to the proportional scale (reference coordinates) used as the reference object in step specially, if the picture is rotated, the cross line can be rotated for correction, and the rotation angle will be recorded by the software: please refer to FIG. 12;

(3) determining a two-dimensional coordinates of the focus, selecting the scanning plane where the target to be positioned is located (the same photo or re-photographed photos can be used), and displaying, the virtual proportional scale on the software again, overlapping the virtual proportional scale with the proportional scale on the current image data by manual or automatic recognition function; calculating, by software to automatically generate a two-dimensional coordinate with the coordinate value of the intersection point as a center by using, the two-dimensional coordinates of the intersection of the cross line in the reference coordinates and the rotation angle of the cross line: at this tinge, the generated two-dimensional coordinate is the measurement coordinate, the position of the measurement coordinate on the target plane is consistent with the position of the above cross line on the baseline plane; please refer to FIG. 12;

(4) determining the two-dimensional coordinate value of the focus; measuring the position of any point in the target plane by using the two-dimensional measurement coordinate generated in step 3, and clicking on any position to display the two-dimensional coordinate values;

S2. firstly drawing a scanning baseline on a body surface; the scanning baseline being an orbitomeatal base line which is a connecting line of an external auditory canal and a midpoint of an eyeball; and then marking two epicranium projection points, a midline and parallel lines beside the midline of a head by using the marking ruler;

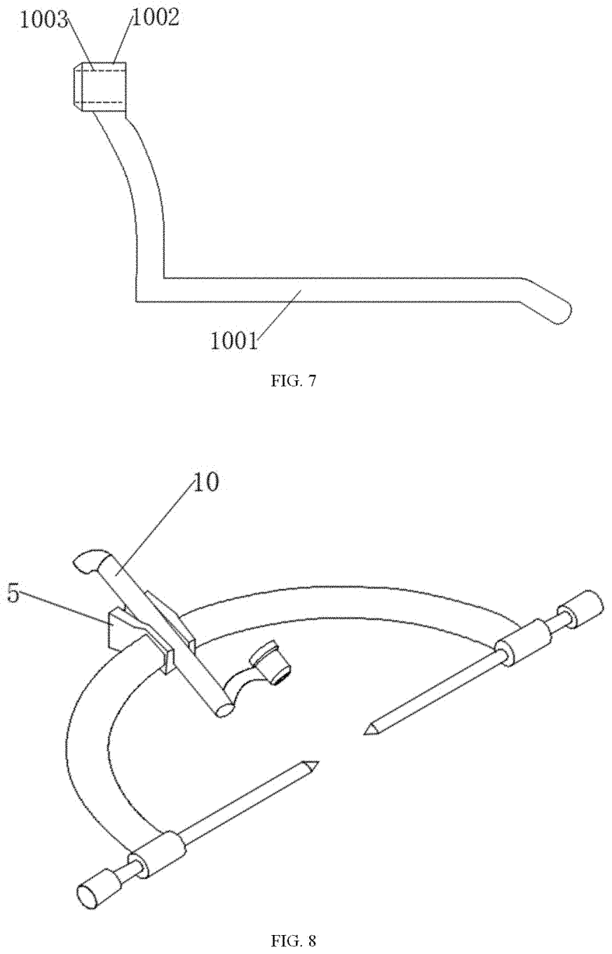

As shown in FIG. 7, the marking ruler can be a fixed marking ruler, which is L-shaped; and the fixed Z-axis ruler 301 and the fixed horizontal ruler 302 are perpendicular to each other. The fixed horizontal ruler 302 is slidably connected with a main slider 303, and each side of the main slider 303 is provided with an carping part for inserting into an ear, and a center point of the earplug part is at an intersection of two scale lines that are perpendicular to each other. When the main slider 303 is pushed to a corner, a center position of the earplug is at the intersection of the scale lines, perpendicular to each other, on the fixed Z-axis ruler 301 and the fixed horizontal ruler 302, namely, Y axis of the fixed horizontal ruler 302 and Z axis of the fixed Z-axis ruler 301.

At the same time, the marking ruler can also be a foldable marking ruler, as shown in FIG. 8. The foldable Z-axis ruler 401 is used as the Z axis, and the foldable horizontal ruler 402 is used as the Y axis. When the foldable marking ruler is unfolded, a notch can ensure that the two axis are vertical. The starting end positions of the scale on the two axis overlap.

The using methods of two marking rulers are similar. The connecting line of the external ear canal and the midpoint of the eyeball is drawed according to the CT or MR film as a baseline. The earplug is slid to the corresponding scale according to the value of the coordinate value given by the App software. Then the earplug is inserted into one side of the external ear canal, the edge of the earplug is adjusted to be parallel to the baseline, and then the distance between the target plane and the reference plane based on the image data as the Z value is obtained. A dot mark is marked with a marker pen on a position corresponding to the scale of the other axle. The marking method, is the same on both sides of the skull. At this time, two points are marked on the epicranium. The two points are the projection of the target point on the epicranium. The midline and the parallel lines located X beside the midline of the skull are then drawn by a ruler.

S3. determining a paracentesis point by using the quarter-bow guide device.

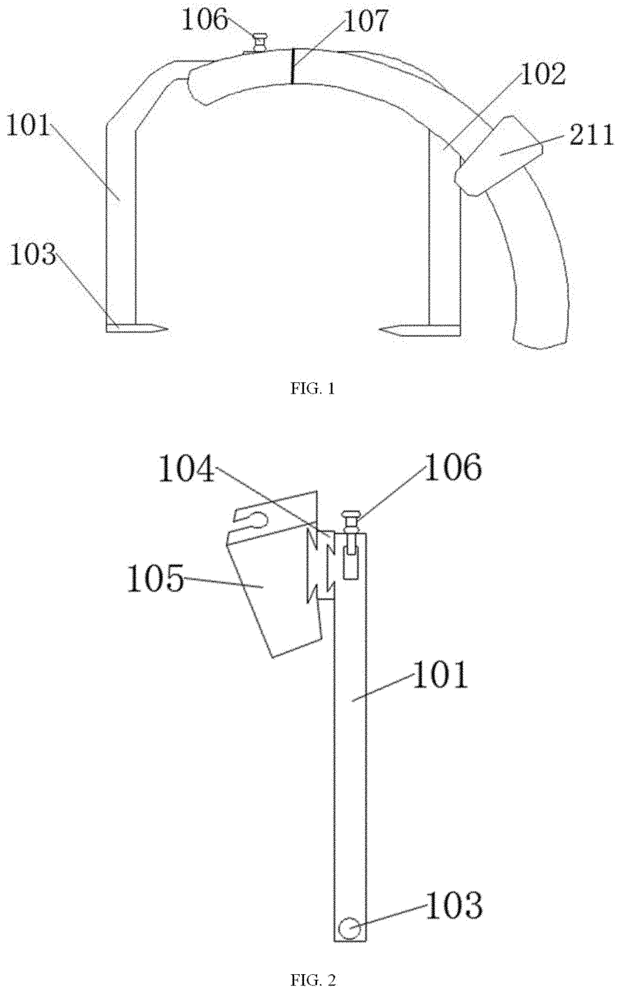

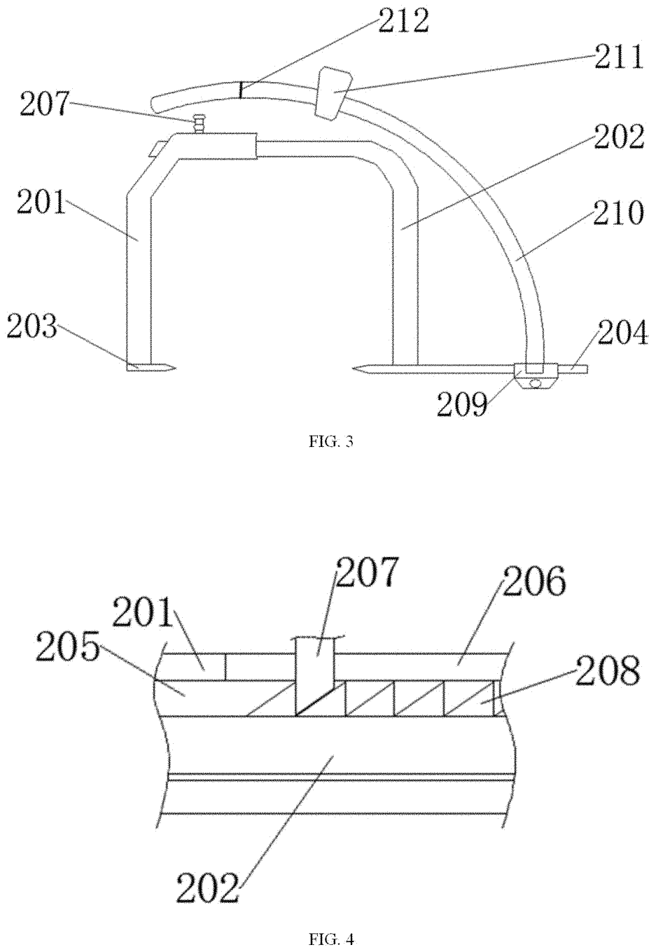

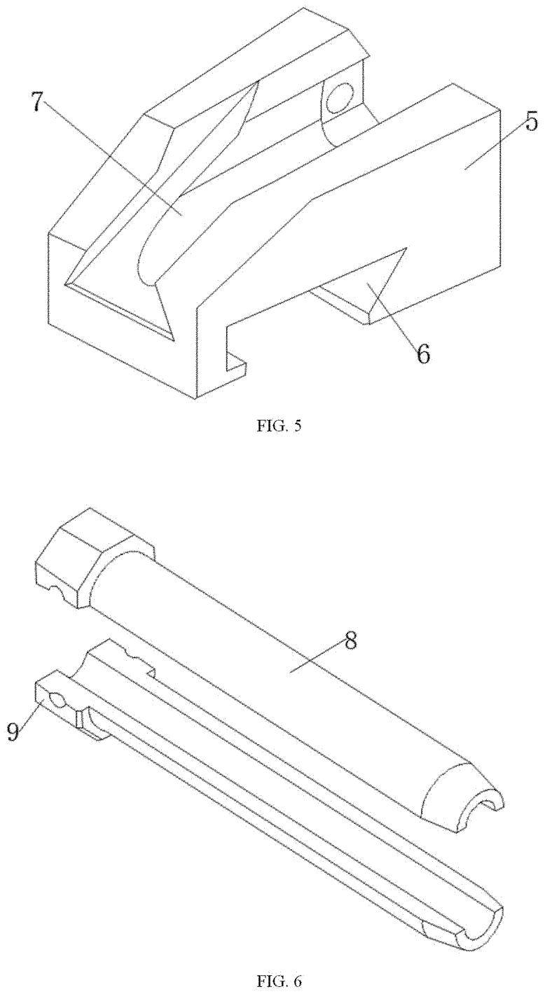

As shown in FIGS. 1-12, the quarter-bow guide device can be the first quarter-bow guide device. The two main head nails 103 are respectively aligned with the two dot marks on both sides of the head, and the arms are clamped. The first main fixing frame 101 and the first accessory fixing frame 102 are fixed on the head, and the main quarter-bow 105 is rotated left and right about the main connecting block 104 as an axis, so that the main zero-degree midpoint 107 is aligned with the parallel lines beside the midline. The main knob 106 is used to fix the position of the main quarter-bow 105. At this time, the positioning direction of the main positioning device 108 always points to the center of the circular bow, that is, the target position, and the depth is the radius of the circular bow.

Similarly, the second quarter-bow guide device can be chosen as the positioning device of the target. The short head nail 203 and the long head nail 204 are respectively aligned with the dot marks on both sides of the head, and the two arms are folded together. The second main fixing frame 201 and the second accessory fixing frame 202 are fixed on the head, and the accessory quarter-bow 210 is moved left and right along the direction of the long head nail 204, so that the accessory zero-degree midpoint 212 is aligned with the parallel lines beside the midline, and paracentesis positioning is performed by the accessory positioning device 211, and the positioning direction always points to the center of the circular bow, that is, the target position, and the depth is the radius of the circular bow.

After the positioning is completed, a pair of guide channels 8 with suitable size can be inserted into the slot 7, and a guide channel 8 with a suitable size is inserted into the half-column groove of the guide channel 8, and the position of the slot 7 on the quarter-bow is adjusted to make the guide channel 8 have the best paracentesis angle, which is convenient for the medical personnel to take the sample biopsy.

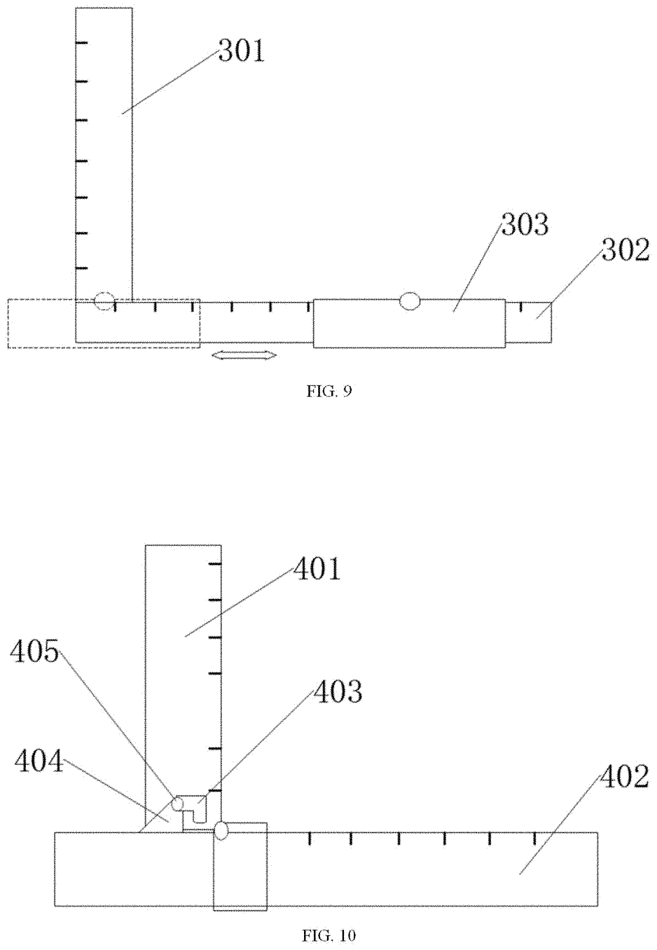

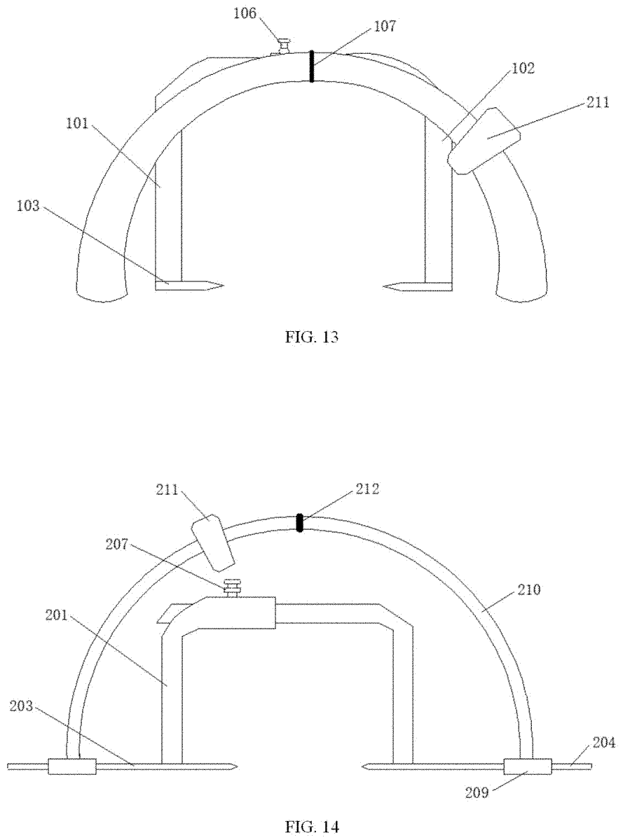

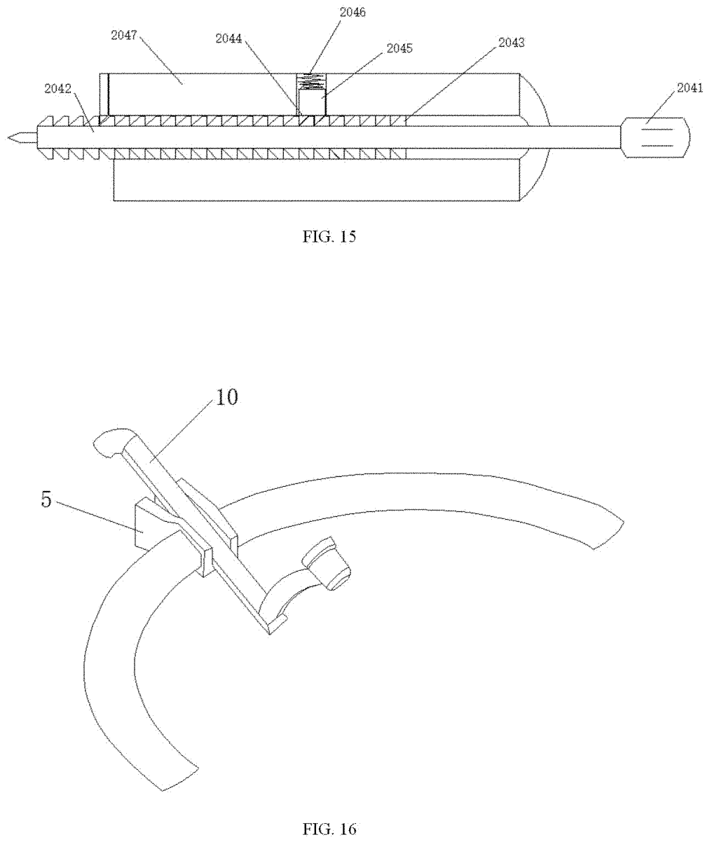

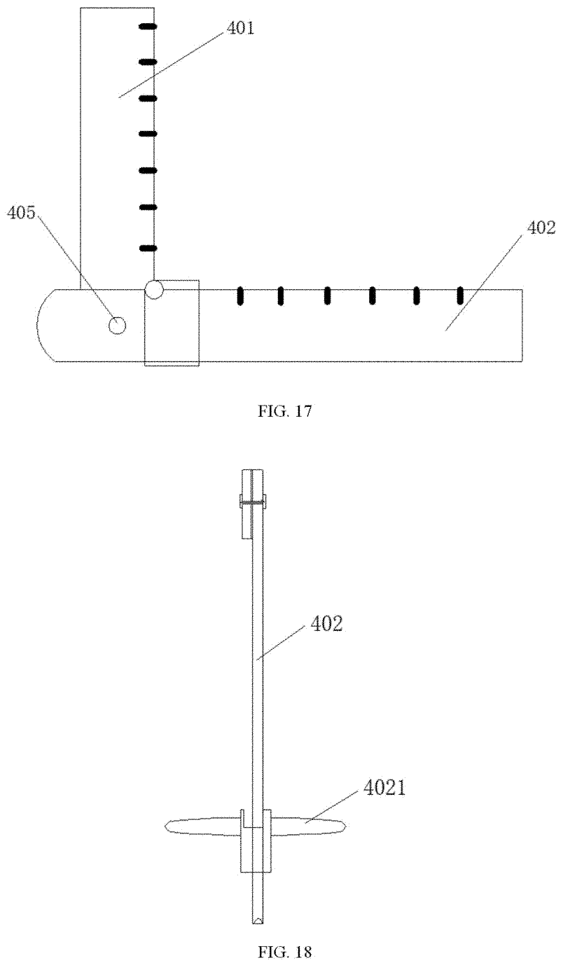

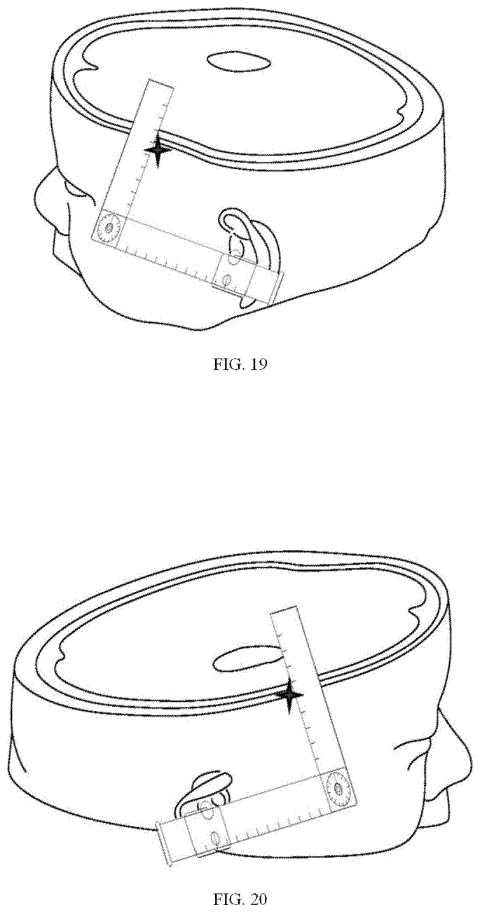

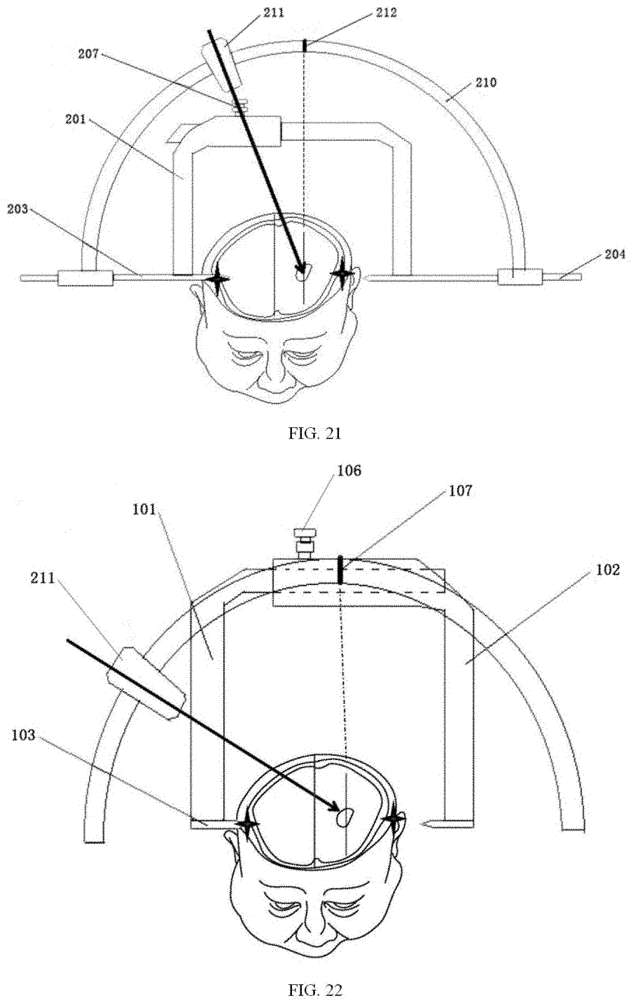

As shown in FIG. 13, a first semicircular-bow guide device can be used for guiding. The difference between the first semicircular-bow guide device and the first quarter-bow guide device is the addition of a main quarter-bow 105, the two main quarter-bows 105 are symmetrical about the main zero-degree midpoint 107, and the other structures are the same as the first quarter-bow guide device. For the target positioning device, as shown in FIG. 14, a second semicircular-bow guide device can be used for positioning. The difference between the second semicircular-bow guide device and the second quarter-bow guide device is that a accessory quarter-bow 210 is added: and two accessory quarter-bows are symmetrical about the accessory zero-degree midpoint 212; and the other structures are the same as that of the second quarter-bow guide device. Please refer to FIG. 15, positioning nails include long head nail 204 and short head nail 203. The long head nail 204 includes a nail body 2042, a rotating head 2041 located at one end of the nail body 2042. A surface of the nail body 2042 has a plurality of sliding teeth 2042. The long head nail 204 further includes an upper wall 2047, a positioning post 2045 embedded in the upper wall 2047. One end of the positioning post 2045 has a fixed tooth 2044, the other end of the positioning post 2045 is connected to a position restoration device 2046. The position restoration device 2046 can produce elastic deformation, on the one hand, it allows the sliding tooth 2042 to move toward the direction to be positioned and be fixed by the positioning post 2045; on the other hand, when the position restoration device 2046 is repositioned, it allows the sliding teeth 2042 to move away from the direction to be position-d to achieve a precise positioning of the long head nail 204. In this embodiment, by turning the rotating head 2041, a fast and rough positioning is first achieved and then a slow and precise positioning is achieved, which greatly improves the positioning efficiency. Please refer to FIG. 16, an end of the extension rod 10 in this embodiment is trapezoidal, and the shape is consistent with that of an internal slot of the positioning block 5, which can ensure the positioning accuracy of the extension rod 10. Please refer to FIGS. 17-20, which provide another foldable marking ruler. Compared with the foldable marking ruler shown in FIG. 9, the only difference is that the connecting shaft 405 is used to connect the foldable Z-axis ruler 401 and the foldable horizontal ruler 402, and the foldable Z-axis ruler 401 can rotate about the connecting shaft 405 by 0-90°. As shown in FIG. 18, each side of the foldable horizontal ruler 402 includes an earplug 4021. Using the patient's ear as a reference plane, the earplug 4021 can be quickly aligned with the patient's ear, such that the precise positioning of the marking ruler is realized. Compared with the marking ruler shown in FIG. 9, the structure is simplified and the operation is more convenient. As shown in FIGS. 19-22, projection mark of the target is drawn on the patient's body through the marking ruler, and the projection mark is used by the first semicircular-bow guide device and the second semicircular-bow guide device to complete the positioning of the focus, and the positioning operation can be completed quickly and accurately, and the positioning cost is greatly reduced.

The above is only preferred embodiments of the present disclosure which do not limit the protection scope of the present disclosure. The equivalent substitution or change made by any person skilled in the art according to the technical solution and improvement concept of the present disclosure shall be included in the protection scope of the present disclosure.

1. A three-dimensional craniocerebral positioning operation tool, comprising a marking ruler and a quarter-bow guide device: wherein the marking ruler comprises a fixed marking ruler; the fixed marking ruler comprises a fixed horizontal ruler (302); and a fixed Z-axis ruler (301) is fixedly connected to one end of the fixed horizontal ruler (302), and the fixed Z-axis ruler (301) and the fixed horizontal ruler (302) are perpendicular to each other; the fixed Z-axis ruler (301) and the fixed horizontal ruler (302) are both engraved with scale marks; zero-scale ends of the fixed Z-axis ruler (301) and the fixed horizontal ruler (302) are located at an intersection of the fixed Z-axis ruler (301) and the fixed horizontal ruler (302); a main slider (303) is sleeved on the fixed horizontal ruler (302), and the fixed horizontal ruler (302) is matched with the main slider (303); the main slider (303) is capable of moving slowly on the fixed horizontal ruler (302) under an action of external force:
the quarter-bow guide device comprises a first quarter bow guide device; and the first quarter-bow guide device comprises a first main fixing frame (101) find a first accessory fixing frame (102); a main square slot that fits the first accessory fixing frame (102) is formed in the first main fixing frame (101), and the first accessory fixing frame (102) is inserted in the main square slot; a main through hole slot is formed in an upper end of the first main fixing frame (101) and a main knob (106) is inserted in the main through hole slot; and upper end of the first accessory fixing frame (102) is fixedly connected with a main serrated block that fits the main knob (106); and the first main fixing frame (101) and the first accessory fixing frame (102) are fixedly connected by the main serrated block and the main knob (106) therebetween; lower ends of the first main fixing frame (101) and the first accessory fixing frame (102) are fixedly connected with main head nails (103): and a side wall of the first main fixing frame (101) is fixedly connected with a main connecting block (104); and one end of the main connecting block (104) far away from the first main fixing frame (101) is fixedly connected with a main quarter-bow (105); and a main positioning device (108) is slidably connected on the main quarter-bow (105); and a main zero-degree midpoint (107) is engraved on the main meeting block (104).

2. The three-dimensional craniocerebral positioning operation tool of claim 1, wherein the marking ruler is a foldable marking ruler, the foldable marking ruler comprises a foldable horizontal ruler (402); one end of the foldable horizontal ruler (402 is connected to a foldable Z-axis ruler (401), and the foldable Z-axis ruler (401) is perpendicular to the foldable Horizontal ruler (402): the foldable Z-axis ruler (401) and the foldable horizontal ruler (402) are both engraved with scale marks; zero-scale ends of the foldable Z-axis ruler (401) and the foldable horizontal ruler (402) are located at an intersection of the foldable Z-axis ruler (101) and the foldable horizontal ruler (402): an L-shaped groove (403) is formed on an end of the foldable Z-axis ruler (401) close to the foldable horizontal ruler (402): an end of the foldable horizontal ruler (402) close to the foldable Z-axis ruler (401) is fixedly provided with two triangular plates (404), and the two triangular plates (404) are respectively located on two sides of the foldable Z-axis ruler (401): a connecting shaft (405) is fixedly connected between the two triangular plates (404), and the connecting shaft (405) penetrates the L-shaped groove (403): the foldable Z-axis ruler (401) and the foldable horizontal ruler (402) are movably connected through the L-shaped groove (403) and the connecting shaft (405).

3. The three-dimensional craniocerebral positioning operation tool of claim 1, wherein the quarter-bow guide device comprises a second quarter-bow guide device, and the second quarter-bow guide device comprises a second main fixing frame (201) and a second accessory fixing frame (202): an accessory square hole slot (205) that fits the second accessory fixing frame (202) is formed in the second main fixing frame (201), and the second accessory fixing frame (202) is inserted into the accessory square hole slot (205); a accessory through hole slot (206) is formed in an upper end of the second, main fixing frame (201), and upper end of the second main fixing frame (201) is connected with an accessory knob (207): and the accessory knob (207) penetrates the accessory through hole slot (206), and an upper end of the second main fixing frame (202) is fixedly connected with an accessory serrated block (208) that matches with the accessory knob (207); lower ends of the second main fixing frame (201) and the second accessory fixing frame (202) are respectively fixedly connected with a short head nail (203) and a long head nail (204): a sliding connection block (209) is slidably connected to the long head nail (204), and the sliding connection block (209) is located at an end of the long head nail (204) away from the short head nail (203); the sliding connection block (209) is fixedly connected with an accessory quarter-bow (210), the accessory quarter-bow (210) is slidably connected with an accessory positioning device (211), an accessory zero-degree midpoint (212) is engraved on the accessory quarter-bow (210); the long head nail (204) is directly inserted into the second accessory fixing frame (202) and is capable of being screwed into and out of the second accessory fixing frame (202), and the long head nail (204) is uniaxially inserted into the second accessory fixing frame (202) from outside to inside; an elastic serrated device fixedly connected to an inner wall of he second accessory fixing frame (202) restricts the long head nail (204) from moving from inside to outside; serration of the elastic serrated device is in an arcuate thread shape: and a cross-section of the serration is a right-angled trapezoid, the serration corresponds to a thread on the main head nail (204) with a cross-section of right-angled trapezoid; the serration limits the main head nail (204) being locked in one direction and serves as a thread, which corresponds to the thread on the main head nail (204), and rotatablely fits to the thread on the main head nail (204).

4. The three-dimensional craniocerebral positioning operation tool of claim 1, wherein an inner diameter and an outer diameter of main quarter-bow (105) and the accessory quarter-bow (210) are 180 mm-300 mm and 220 mm-400 mm respectively: and a width of the main quarter-bow (105) and the accessory quarter-bow (210) is 20 mm-50 mm.

5. The three-dimensional craniocerebral positioning operation tool of claim 1, wherein a material of the main quarter-bow (105) and the accessory quarter-bow (210) is selected from metal, plastic and polymer material: and tips of the main quarter-bow (105) and the accessory quarter-bow (210) are polished smoothly.

6. The three-dimensional craniocerebral positioning operation tool of claim 1, wherein the main positioning device (108) and the accessory positioning device (211) have same structure: each of the main positioning device (108) and the positioning devices (211) comprises a positioning block (5): one end of the positioning block (5) is engraved with a rotational sliding groove (6); and the positioning block (5) is connected to the main quarter-bow (105) or the accessory quarter-bow (210) through the rotational sliding groove (6); and a slot (7) is formed at an end of the positioning block (5) away from the rotational sliding groove (6).

7. The three-dimensional craniocerebral positioning operation tool of claim 6, wherein an extension rod (10) is inserted into the slot (7): and the extension rod (10) comprises an L-shaped rod body (1001); the L-shaped rod body (1001) comprises a main body part and an extension part; the extension part of the L-shaped rod body (1001) is fixedly connected with a plug-in block (1002), and a positioning hole (1003) is firmed in the plug-in block (1002): and an axis of the positioning hole (1003) is parallel to the main body part of the L-shaped rod body (1001).

8. The three-dimensional craniocerebral positioning operation tool of claim 7, wherein a pair of guide channels (8) matches with each other are inserted into the positioning hole (1003), and one end of the guide channel (8) is fixedly connected a limited block (9).

9. The three-dimensional craniocerebral positioning operation tool of claim 8, wherein the guide channel (8) is inserted into a half-column groove, and the guide channel (8) has a variety of models, and different models of the half-column groove have different diameters.

10. A using method of the three-dimensional craniocerebral positioning operation tool of claim 1, comprising:
S1. filming CT or NM data images, and transmitting the data images obtained to a computer or other processing terminal, and transferring the data images to corresponding processing software;
S2. establishing a reference coordinate with a location and scaling of a scale on the CT or MR data images as a reference; determining a measurement coordinate by the reference coordinate; defining a position of a target point in the measurement coordinate; taking pictures of the CT or MR data images by using photographing and calculation functions of mobile phones and other electronic devices; calculating coordinate values of the target point of a focus by automatically processing to information of the pictures; and marking the coordinate values as (X, Y, Z);
S3. firstly drawing a scanning baseline on a body surface; the scanning baseline being an orbitomeatal base line which is a connecting line of an external auditory canal and a midpoint of an eyeball; and then marking two epicranium projection points, a midline of a head and parallel lines beside the midline by using the marking ruler;
S4. determining a paracentesis point by using the quarter-bow guide device.
